(19) United States
(12) Patent Application Publication
(10) Pub. No.: US 2011/0118220 A1
(43) Pub. Date: May 19, 2011

(54) Composition of bifonazole and its use

(75) Inventors: Jason Xing Zeng, Burlingame, CA (US); Lingson Dai, Burlingame, CA (US)

(73) Assignee: Fibonacci Biological Medicine, LLC

(21) Appl. No.: 12/590,748

(22) Filed: Nov. 13, 2009

(51) Int. Cl. A61K 31/56

(52) U.S. Cl. 514/171

(57) ABSTRACT

A medical composition includes Bifonazole and Triamcinolone acetonide acetate, and is mixed with pharmaceutical acceptable accessory. The medical composition of the present invention is used in treatment of skin superficial fungal infections, and also has the dual role of anti-inflammatory, anti-allergy and anti-itching functions.

### BACKGROUND OF THE PRESENT INVENTION

1. Field of Invention

The present invention relates to a medicine composition comprising bifonazole and more particularly relates to a medicine composition comprising anti-superficial fungal bifonazole and anti-inflammatory, anti-allergic and antipruritic triamcinolone acetonide acetate, mainly for the treatment of superficial mycoses.

2. Description of Related Arts

Dermatologists point out that glucocorticoid hormones combined with antifungal is useful in treating of fungal infected skin disease and similar diseases. Clinical studies also showed that antifungal medicine and glucocorticoid hormones medicine combination can accelerate disease remission and improve fungicide effect. Accordingly, the present invention provides an anti-inflammatory, anti-allergic and antipruritic medicine comprising bifonazole as antifungal and triamcinolone acetonide acetate as an anti-inflammatory, anti-allergy and antipruritic medicine.

Bifonazole is an imidazole derivative. It has a broad antimicrobial spectrum for inhibition 90% of pathogenic fungi. It can inhibit a variety of skin fungi (such as Trichophyton, Microsporum), yeast (such as Candida, bottle-shaped yeast, Torulopsis glabrata), mold (such as Aspergillus), and a variety of fungi (such as Coccidioides, Blastomyces, Histoplasma). It also has some gram-positive bacteria (such as Staphylococcus, Streptococcus, etc.) activity. The inhibitory mechanism of bifonazole is: preventing the synthesis of the membrane lipid composition ergosterol in low concentration, and changing the nature of the membrane lipid specific binding membrane in high concentration, so as to dysfunction the cell membrane of the fungi. Usually Bifonazole can be applied locally for treatment of superficial fungal diseases such as tinea cruris, athlete's foot, hand tinea, candidiasis, pityriasis versicolor, and onychomycosis. Some patients' symptom of rosacea, psoriasis, Sebopsoriasis and seborrheic dermatitis is also found improved (H F Doering, Dermatologica 169 (suppl):125-134, 1984; GP Ford, et al., ibid169 (suppl): 135-140, 1984).

Triamcinolone acetonide acetate has anti-inflammatory, anti-allergy and anti-itching effect by applied externally, and the effect is strong and durable. It can eliminate the topical non-infectious inflammation caused by fever, redness and swelling. It is clinically used in allergic dermatitis, eczema, neurodermatitis, seborrheic dermatitis and pruritus.

Bifonazole and Triamcinolone acetonide acetate are developed as separate preparation. There are also compound preparation containing both anti-fungal drugs and hormone drugs. But there is no compound preparation comprising Bifonazole and Triamcinolone acetonide acetate because not all of the anti-fungal drugs and the hormone drugs composition can cooperate together. By doing a lot of research, screening the composition of these two types of drugs in many conditions, analyzing a large amount of data, we determined the composition efficacy, pharmacology and toxicology, found out the dual role of anti-inflammatory activity and rapidly alleviate the symptoms of fungal infection. In the present application we provide a medical composition comprising effective amount of Bifonazole and Triamcinolone acetonide acetate with high efficiency and duration.

### SUMMARY OF THE PRESENT INVENTION

An object of the present invention is to provide a medical composition comprising Bifonazole.

Accordingly, in order to accomplish the above object, the present invention provides a composition of Bifonazole, comprising Bifonazole, Triamcinolone acetonide acetate, and pharmaceutical acceptable accessory.

Accordingly, the composition of Bifonazole comprises 0.01˜2% Bifonazole, 0.01˜0.5% Triamcinolone acetonide acetate, and pharmaceutical acceptable accessory as the rest.

The present invention further provides a method of producing a medicine composition for skin infections especially in treatment of skin superficial fungal infections, comprising the steps of:

(a) mixing 0.01 to 2% of Bifonazole with 0.01 to 0.5% of Triamcinolone acetonide acetate to form a first mixture;

(b) mixing a mixture of Bifonazole and Triamcinolone with a pharmaceutical acceptable accessory to form a final product, and

(c) encapsulating and packing the final product.

In order to accomplish the above object, the composition of Bifonazole is for external use.

In order to accomplish the above object, the pharmaceutical acceptable accessory is added during the preparation of the composition.

The usage of the present invention of the composition of Bifonazole in treatment of skin superficial fungal infections:

1. Pharmacodynamic Study

To understand the in vivo and in vitro antibacterial effects, and to determine the in vivo and in vitro antibacterial activity through measuring the compound Bifonazole cream (Bifonazole/Triamcinolone acetonide acetate) on common pathogenic fungi of the minimum inhibitory concentration (MIC).

The In Vivo Test Results:

(1) applying compound bifonazole cream on animal topically on skin infections caused by Gypsum-like Trichophyton rubrum, can significantly reduce the degree of topical skin lesions, improve negative rate of Gypsum-like Trichophyton rubrum. It can significantly reduce the degree of topical skin lesions caused by Trichophyton rubrum, and has significant effect on superficial fungal infections.

(2) compound Bifonazole cream significantly increases the anti-itching threshold of guinea pigs on histamine phosphate, and has a significant anti-itching effect.

(3) the effect of the (10:1) set of compound Bifonazole cream is much better than the (5:1) set, and much stronger than unilateral Bifonazole, but has no significant difference from Pevisone.

The in vitro experiment results (through nine kinds of antifungal agents against 543 common fungi in vitro pharmacodynamics):

(1) Bifonazole has a strong antibacterial activity on filamentous fungi represented by Trichophyton rubrum, has a certain antibacterial activity on candida such as candida Albicans.

(2) The antimicrobial activity of Bifonazole against common fungal skin infections is slightly stronger than econazole, ketoconazole, clotrimazole and miconazole.

(3) Bifonazole and Triamcinolone acetonide acetate in ratio of 10:1 has no in vitro antibacterial activity.

2. Pharmacological Study

To provide a comprehensive a evaluation of the efficacy and safety of the Bifonazole cream by observing effects of Bifonazole on whole animals, nervous system, cardiovascular and respiratory systems.

Effects on Cardiovascular System

Cat cardiovascular and respiratory test shows that, applying of 2, 1, and 0.5 g/kg compound Bifonazole cream respectively on cat's skin has not obvious effect on cat's cardiovascular system and respiratory system. There is no significant difference on mean arterial blood pressure, respiratory rate, respiratory frequency and amplitude, heart rate, QRS wave group, ST segment, T wave of ECG compared with excipients (P>0.05).

Effects on Nervous System

The general behavior and autonomous activities of mice shows that, applying of 4, 2, and 1 g/kg compound Bifonazole cream respectively on mice's skin has not obvious effect on mice's nervous system. There is no adverse impact on any observation indicator such as general behavior, pupil, posture, gait, salivation, and muscle trembling, and on Spontaneous activity compared with excipients (P>0.05).

Experiment Result Shows:

(1) By applying compound Bifonazole cream on the skin of mice, the mice's general behavior, pupil, posture, and gait are in normal. No salivation and muscle trembling are observed. The reaction is the same as the control mice. There is no significant difference among mice with different dosage and control mice (P>0.05).

(2) By applying of 2, 1, and 0.5 g/kg compound Bifonazole cream respectively on cat's naked skin, no difference on blood pressure. Compared with control group with excipient, the difference is not obvious (P>0.05). Three dosages have no significant effect on cat's electrocardiogram, respiratory rate, and respiration depth (amplitude), and no significant difference from control group with excipient.

According to the above experiment results, compound Bifonazole cream of 2, 1, and 0.5 g/kg dosages have no significant effect on mice nervous system. 2, 1, and 0.5 g/kg dosages have no significant effect on cat's breathing, respiratory amplitude, blood pressure, heart rate, ECG, etc. In the experiment conditions, the compound Bifonazole scream doesn't show significant effect on cat's cardiovascular system and respiratory system.

3. Acute Toxicity Study

Observe the toxicity reaction by smearing Bifonazole on mice and gavage feeding of Bifonazole to mice.

Experiment Result Shows:

After gavage feeding once, no mouse die in 7 days. In histopathological observation, after 7 days, the mice were killed and found that the liver of mice turned into dark purple. In microscopic observation, the liver was significantly cloudy swelling, massive cytoplasm, cell body increased, sinusoid is unclear, showed swelling-like cloudy. Other tissues and organs had no significant changes. Mice skin was smeared by compound Bifonazole. The maximum dosage is 4 g/kg. The dosage is about 235.3 times of human clinical dosage. Oral feeding mice compound Bifonazole 0.4 ml/kg (body weight), although 20 mice were all survived, the weight of mice is limited, the weight gain is suppressed. In anatomic visual observation, the color of the liver of mice changed to dark purple. In microscopic observation, the liver had severe hepatic necrosis.

4. Experimental Study on Long-Poisoning

Observe the abnormal reaction and degree of reversibility of the reaction caused by skin penetration through long-term exposure the animal's skin to subject drug.

High-dose group of compound Bifonazole cream has a certain toxicity to the liver (due to rats eating the cream by mistake), but no significant toxicity for recovery.

High dose of compound Bifonazole cream has a certain skin irritation; middle dosage group has mild skin irritation, but has no significant toxicity on various organs; low dosage has no significant toxicity on various organs.

The minimum toxic dosage of Bifonazole on main target organs in the 2 g/kg dose in the liver should be 2 g/kg.

Compound Bifonazole cream comprises Bifonazole and Triamcinolone acetonide acetate, both are reported to have toxicity.

(1) Compound Bifonazole cream has no significant impact on rat's body weight, has no toxicity on rat's behavior, activity, fur, walking posture, and mental systems; has no significant impact on rat's feeding and water drinking.

(2) Compound Bifonazole cream has no significant impact on rat's routine blood test indicators; has no significant impact on rat's blood biochemistry indicators except AST and T-Bil increasing.

(3) Compound Bifonazole cream has no significant impact on rat's organ coefficient and pathological histology, but in high dosage group, individual rat livers (2 out of 10) have symptoms such as massive liver cell cytoplasm, cell body increased, sinusoid is unclear, cloudy swelling shape, skin inflammatory cell infiltration, surface hyperkeratosis and edema-like changes, has no significant effect on other organs.

5. Allergy, Local Irritation Experimental Study

Observe the immune system response on skin after repeating skin contact animal skin with testing object.

(1) Allergy Study

Exipients Group

The average response is 0 by instance, 24 hours, 48 hours, and 72 hours individually. No erythema and edema formed. Allergenic rate is 0%.

Drugs Group (Compound Bifonazole Cream)

The average response is 0 by instance, 24 hours, 48 hours, and 72 hours individually. No erythema and edema formed. Allergenic rate is 0%.

Positive allergen group (2,4-dinitro-chlorobenzene)

The average response is 2.4 by instance and 24 hours, 2.7 by 48 hours, 2.9 by 72 hours individually. It is between moderate and severe allergic reaction.

Allergenic rate: in 2,4-dinitro-chlorobenzene group, 10 animals all have erythema and edema. The allergenic rate is 100%. It has extremely high allergenicity while the excipients and the drug group have weak allergenicity.

(2) Local Irritation Study

Smear 1.0 g compound bifonazole cream to intact skin and damaged skin of rabbits each with sufficient contact, fixed with gauze and non-stimulated ventilation tape, rear in single cage, wash out testing object after 24 hours, observe by 1, 24, 48, 72 hours individually: intact skin has no irritation, damaged skin is thickened and uplifted. The skin irritation evaluation criteria scoring is about 1.5, which is mild stimulation. Skin histopathological examination shows edema-like loose dermis, and bleeding like red blood cells, epidermal hyperplasia is observed.

Compared with the existing technology, the beneficial effect of this invention is:

(1) The glucocorticoid hormones and antifungal combination has been reported, but it is only a brief, qualitative description of its benefit in treatment of fungal infections of the skin and similar diseases. The present invention provides a composition consisting Bifonazole as antifungal drug and Triamcinolone acetonide acetate as anti-inflammatory, anti-allergic and antipruritic drug. It is a practical research in this model of drug composition, and provides quantitative results in treatment of skin fungal infections and similar diseases which has a strong guiding significance.

(2) Pharmacodynamics experimental studies have shown that the present invention had a surprisingly good result. Compound Bifonazole cream local administration had a significant antibacterial effect on superficial mycoses plaster-like Trichophyton rubrum, skin infection caused by Trichophyton rubrum, significantly reduced the infection and damage of the skin, promoted restoration of normal skin, and markedly increased the histamine phosphate itch. High dose of compound Bifonazole cream has certain irritation on rat's skin, middle dose of compound Bifonazole cream has limited irritation but has no significant toxicity on organs, low dose has no significant toxicity on skin and the organs.

(3) The pharmaceutical compositions of this invention has done a comprehensive study in fields of composition, preparation techniques, efficacy, pharmacology, toxicology, etc. The study is sufficient, and has a strong novelty.

These and other objectives, features, and advantages of the present invention will become apparent from the following detailed description, the accompanying drawings, and the appended claims.

### BRIEF DESCRIPTION OF THE DRAWINGS

FIG. 1 is a table showing Therapeutic effect on Gypsum-like Trichophyton rubrum infections.

FIG. 2 is a table showing Effect on Gypsum-like Trichophyton rubrum infections.

FIG. 3 is a table showing Therapeutic effect on Gypsum-like Trichophyton rubrum infections.

FIG. 4 is a table showing Compound Bifonazole cream anti-itching effect.

### DETAILED DESCRIPTION OF THE PREFERRED EMBODIMENT

According to a present invention, a medicine composition is provided for skin infections especially in treatment of skin superficial fungal infections and also has the dual role of anti-inflammatory, anti-allergy and anti-itching functions, wherein the medicine composition comprises a mixture of 0.01 to 2% of Bifonazole, 0.01 to 0.5% of Triamcinolone acetonide acetate, and a pharmaceutically acceptable accessory.

Accordingly, a method of producing the medicine composition for skin infections especially in treatment of skin superficial fungal infections, comprises the following steps.

(1) Mix 0.01 to 2% of Bifonazole with 0.01 to 0.5% of Triamcinolone acetonide acetate to form a first mixture.

(2) Mix the first mixture of Bifonazole and Triamcinolone with a pharmaceutical acceptable accessory to form a final product.

(3) Encapsulate and pack the final product.

According to the first embodiment, the medicine composition is formed in cream form for externally applying on a human skin, wherein the weight ratio of Bifonazole, Triamcinolone acetonide acetate, and the pharmaceutically acceptable accessory is approximately 10:1:391.

In particularly, the pharmaceutically acceptable accessory comprises 1,2-propanediol, Stearic acid, Hexadecanol, Liquid paraffin, Glyceryl monostearate, Sodium lauryl sulfate, and Ethylparaben. In addition, the weight ratio of 1,2-propanediol, Stearic acid, Hexadecanol, Liquid paraffin, Glyceryl monostearate, Sodium lauryl sulfate, and Ethylparaben is 80:60:60:120:60:10:1.

The formulation for preparing 1000 units of the medicine composition in cream form, each unit of the medicine composition containing 100 mg of Bifonazole, and 10 mg of Triamcinolone acetonide acetate, is illustrated as follows:

| | |
|---|---|
| Bifonazole | 100 g |
| Triamcinolone acetonide acetate | 10 g |
| 1,2-propanediol | 800 g |
| Stearic acid | 600 g |
| Hexadecanol | 600 g |
| Liquid paraffin | 1200 g |
| Glyceryl monostearate | 600 g |
| Sodium lauryl sulfate | 100 g |
| Ethylparaben | 10 g |
| Add water to | 10000 g |

According to the first embodiment, the step (1) further comprises the following steps.

(1.1) Add Bifonazole and Triamcinolone acetonide acetate to propanediol.

(1.2) Heat a mixture of Bifonazole, Triamcinolone acetonide acetate, and propanediol to dissolve to form the first mixture.

According to the method, the step (2) further comprises the following steps.

(2.1) Mix Hexadecanol, liquid paraffin, stearic acid, and glyceryl monostearate together to form a second mixture, heating up the second mixture to 80° C. to melt, and maintain its temperature.

(2.2) Add Sodium lauryl sulfate to deionized water to form a third mixture and heat the third mixture to 80° C. to dissolve.

(2.3) Add the first mixture to the second mixture with stirring to form a cream form mixture.

(2.4) Add the third mixture to the cream form mixture and stirring continuously the third mixture with the cream form mixture to cool to obtain the final product.

According to the second embodiment, the medicine composition is formed in gel form for externally applying on a human skin, wherein the weight ratio of Bifonazole, Triamcinolone acetonide acetate, and the pharmaceutically acceptable accessory is approximately 200:1:7550.

In particularly, the pharmaceutically acceptable accessory comprises Carbopol 980NF, 1,2-propanediol, Deionized water, Polyethylene glycol 400 (PEG 400), and Triethanolamine. In addition, the weight ratio of Carbopol 980NF, 1,2-propanediol, said Deionized water, and Polyethylene glycol 400 (PEG 400) is approximately 15:200:240:300.

The formulation for preparing 1000 units of the medicine composition in gel form, each unit of the medicine composition containing 200 mg of Bifonazole, and 1 mg of Triamcinolone acetonide acetate, is illustrated as follows:

| | |
|---|---|
| Bifonazole | 200 g |
| Triamcinolone acetonide acetate | 1 g |
| Carbopol 980NF | 150 g |
| 1,2-propanediol | 2000 g |
| Deionized water | 2400 g |
| Polyethylene glycol 400 (PEG 400) | 3000 g |
| Triethanolamine | Appropriate value |
| Add Ethanol to | 10000 g |

According to the second embodiment, the step (1) further comprises the following steps.

(1.1) Mix Bifonazole, Triamcinolone acetonide acetate, and a first half amount of polyethylene glycol 400 and propylene glycol with a predetermined amount of anhydrous ethanol to form the first mixture.

(1.2) Heat the first mixture to dissolve.

According to the method, the step (2) further comprises the following steps.

(2.1) Form a second mixture by rapidly stirring Carbopol 980NF and slowly add with deionized water until the second mixture fully swells.

(2.2) Add another half amount of polyethylene glycol 400 and propylene glycol into the second mixture to form a third mixture, and stir the third mixture uniformly.

(2.3) Add and stir a predetermined amount of ethanol solution of triethanolamine with the third mixture to form a clear, transparent Carbopol gel.

(2.4) Slowly add the first mixture into the clear, transparent Carbopol gel, and add and stir the ethanol to prescribed weight to form the final product.

According to the third embodiment, the medicine composition is formed in cream form for externally applying on a human skin, wherein the weight ratio of Bifonazole, Triamcinolone acetonide acetate, and the pharmaceutically acceptable accessory is approximately 1:50:4300.

In particularly, the pharmaceutically acceptable accessory comprises Stearic acid, Glyceryl monostearate, Glycerol, White Vaseline, and Ethyl Hydroxybenzoate. In addition, the weight ratio of Stearic acid, Glyceryl monostearate, Glycerol, and White Vaseline is approximately 10:10:15:8.

The formulation for preparing 1000 units of the medicine composition in cream form, each unit of the medicine composition containing 1 mg of Bifonazole, and 50 mg of Triamcinolone acetonide acetate, is illustrated as follows:

| | |
|---|---|
| Bifonazole | 1 g |
| Triamcinolone acetonide acetate | 50 g |
| Stearic acid | 1000 g |
| Glyceryl monostearate | 1000 g |
| Glycerol | 1500 g |
| White Vaseline | 800 g |
| Ethyl Hydroxybenzoate | Appropriate value |
| Add water to | 10000 g |

According to the third embodiment, the step (1) further comprises the following steps.

(1.1) Mix Bifonazole and Triamcinolone acetonide acetate with a mixture of glycerol, white Vaseline, and ethyl hydroxybenzoate to form the first mixture.

(1.2) Heat the first mixture until its dissolves.

According to the method, the step (2) further comprises the following steps.

(2.1) Mix stearic acid and glyceryl monostearate to form a second mixture, and heat up the second mixture at 80° C. and maintain its temperature.

(2.2) Add and stir the first mixture into the second mixture to form a cream form mixture.

(2.3) Add water into the cream form mixture and continuously stir the cream form mixture until the cream form mixture is cooled down to form the final product in cream form.

The present invention of compound Bifonazole cream (bifonazole—triamcinolone acetonide acetate composition) and the commercial products bifonazole cream (Germany Bayer Healthcare Co., Ltd. production) were compared.

(1) Therapeutic Effect

1. Therapeutic Effect on Gypsum-Like Trichophyton Rubrum Infections

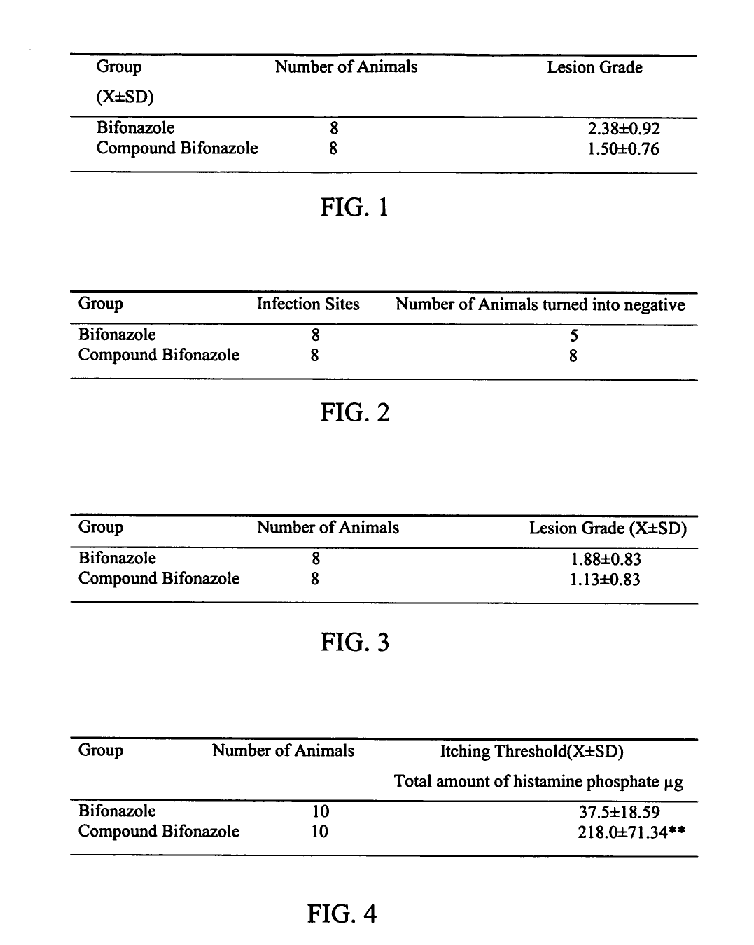

Take 8 health white guinea pigs of either sex. The day before the experiment, remove the hair of both back sides 4×4 cm², and abrade the skin with sterile sandpaper by 1.5×1.5 cm². Apply 20 μl 108 CFU/ml bacterial suspension to the broken organizations uniformly. After 7 days of infection, apply drugs. Group white guinea pigs randomly according to the damage area and dander distribution. Each group contains 4 white guinea pigs (8 sites). Each white guinea pig is applied with the same drug on two sites. Observe the lesion site of infection evaluate for 14 days continuously, compare the difference between groups with the t test, the results are shown in FIG. 1.

Culture a little dander of the infection sites to calculate the negative rate of gypsum-like Trichophyton rubrum; compare the difference between groups with the x² test, the results are shown in FIG. 2.

FIGS. 1 and 2 show that, compound Bifonazole has significant therapeutic effect on Gypsum-like trichophyton rubrum infection. Its therapeutic effect is much better than unilateral Bifonazole.

2. Therapeutic Effect on T. rubrum Infections

Take 8 health white guinea pigs of either sex. The day before the experiment, remove the hair of both back sides 4×4 cm², and abrade the skin with sterile sandpaper by 1.5×1.5 cm². Apply 20 μl 108 CFU/ml T. rubrum suspension to the broken organizations uniformly. After 7 days of infection, apply drugs. Group white guinea pigs randomly according to the damage area and dander distribution. Each group contains 4 white guinea pigs (8 sites). Each white guinea pig is applied with the same drug on two sites. Observe the lesion site of infection evaluate for 14 days continuously, compare the difference between groups with the t test, the results are shown in FIG. 3.

FIG. 3 shows that, compound Bifonazole has significant therapeutic effect on T. rubrum infection. Its therapeutic effect is much better than unilateral Bifonazole.

(2) Antipruritic Effect

Take 20 health white guinea pigs, weight from 200 to 300 g. Divided into two groups of 10 randomly which are (1) Bifonazole group and (2) Compound Bifonazole group. The day before the experiment, shave the hair of right rear feet, administer drug once. On the experimental day, scratch 1 cm² shaved area by raving paper. Administer drug topically again. The control group was given equivalent excipients. 10 minutes after the last administration, began to drop in 0.01% phosphoric acid histamine 0.05 ml/animal at the wound. After every 3 minutes followed by 0.01%, 0.02%, 0.03%, 0.04% . . . increase the concentration in 0.05 ml/animal until the guinea pig start to lick the right rear foot. Use the histamine phosphate given to the guinea pigs when the last licking of the right rear foot was seen as the itching threshold, record and compare between groups. The results are shown in FIG. 4.

FIG. 4 shows that, compared with unilateral Bifonazole, compound Bifonazole significantly increases the itching threshold and has significant anti-itching effect.

The above experiments show that, (1) local administration of compound Bifonazole to animals can significantly reduce the lesions caused by the gypsum-like Trichophyton rubrum skin infection, and improve the negative rate of gypsum-like Trichophyton rubrum; significantly reduce the lesions caused by the T. rubrum skin infection, and effective in anti-superficial mycoses. (2) Compound Bifonazole significantly increases the guinea pig's anti-itching threshold on histamine phosphate, has significant anti-itching effect.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A medicine composition for skin infections especially in treatment of skin superficial fungal infections, comprising a mixture of 0.01 to 2% of Bifonazole, 0.01 to 0.5% of Triamcinolone acetonide acetate, and a pharmaceutically acceptable accessory.

2. The medicine composition, as recited in claim 1, which is formed in cream form for externally applying on a human skin, wherein said pharmaceutically acceptable accessory comprises 1,2-propanediol, Stearic acid, Hexadecanol, Liquid paraffin, Glyceryl monostearate, Sodium lauryl sulfate, and Ethylparaben.

3. The medicine composition, as recited in claim 2, wherein a weight ratio of said Bifonazole, said Triamcinolone acetonide acetate, and said pharmaceutically acceptable accessory is approximately 10:1:391.

4. The medicine composition, as recited in claim 3, wherein a weight ratio of said 1,2-propanediol, said Stearic acid, said Hexadecanol, said Liquid paraffin, said Glyceryl monostearate, said Sodium lauryl sulfate, and said Ethylparaben is 80:60:60:120:60:10:1.

5. The medicine composition, as recited in claim 1, which is formed in gel form for externally applying on a human skin, wherein said pharmaceutically acceptable accessory comprises Carbopol 980NF, 1,2-propanediol, Deionized water, Polyethylene glycol 400 (PEG 400), and Triethanolamine.

6. The medicine composition, as recited in claim 5, wherein a weight ratio of said Bifonazole, said Triamcinolone acetonide acetate, and said pharmaceutically acceptable accessory is approximately 200:1:7550.

7. The medicine composition, as recited in claim 6, wherein a weight ratio of said Carbopol 980NF, said 1,2-propanediol, said Deionized water, and said Polyethylene glycol 400 (PEG 400) is approximately 15:200:240:300.

8. The medicine composition, as recited in claim 1, which is formed in cream form for externally applying on a human skin, wherein said pharmaceutically acceptable accessory comprises Stearic acid, Glyceryl monostearate, Glycerol, White Vaseline, and Ethyl Hydroxybenzoate.

9. The medicine composition, as recited in claim 8, wherein a weight ratio of said Bifonazole, said Triamcinolone acetonide acetate, and said pharmaceutically acceptable accessory is approximately 1:50:4300.

10. The medicine composition, as recited in claim 9, wherein a weight ratio of said Stearic acid, said Glyceryl monostearate, said Glycerol, and said White Vaseline is approximately 10:10:15:8.

11. A method of producing a medicine composition for skin infections especially in treatment of skin superficial fungal infections, comprising the steps of:
(a) mixing 0.01 to 2% of Bifonazole with 0.01 to 0.5% of Triamcinolone acetonide acetate to form a first mixture;
(b) mixing said first mixture of said Bifonazole and said Triamcinolone with a pharmaceutical acceptable accessory to form a final product, and
(c) encapsulating and packing said final product.

12. The method, as recited in claim 11, wherein said pharmaceutically acceptable accessory comprises 1,2-propanediol, Stearic acid, Hexadecanol, Liquid paraffin, Glyceryl monostearate, Sodium lauryl sulfate, and Ethylparaben.

13. The method, as recited in claim 12, wherein the step (a) further comprises a step of adding said Bifonazole and said Triamcinolone acetonide acetate to propanediol and heating a mixture thereof to dissolve to form said first mixture, wherein the step (b) further comprises the steps of:
(b.1) mixing said Hexadecanol, said liquid paraffin, said stearic acid, and said glyceryl monostearate together to form a second mixture, heating up said second mixture to 80° C. to melt, and maintaining its temperature;
(b.2) adding said Sodium lauryl sulfate to said deionized water to form a third mixture and heating said third mixture to 80° C. to dissolve;
(b.3) adding said first mixture to said second mixture with stirring to form a cream form mixture; and
(b.4) adding said third mixture to said cream form mixture and stirring continuously said third mixture with said cream form mixture to cool to obtain said final product.

14. The method, as recited in claim 13, wherein 100 g of Bifonazole, 10 g of Triamcinolone acetonide acetate; 800 g of 1,2-propanediol; 600 g of stearic acid; 600 g of Hexadecanol; 1200 g of liquid paraffin; 600 g of glyceryl monostearate; 100 g of sodium lauryl sulfate; 10 g of Ethylparaben and 5980 g of water are used to make 1000 units of said final product in cream form, wherein each unit of said final product contains 100 mg of Bifonazole and 10 mg of Triamcinolone acetonide acetate.

15. The method, as recited in claim 11, wherein said pharmaceutically acceptable accessory comprises Carbopol 980NF, 1,2-propanediol, Deionized water, Polyethylene glycol 400 (PEG 400), and Triethanolamine.

16. The method, as recited in claim 15, wherein the step (a) further comprises a step of mixing said Bifonazole, said Triamcinolone acetonide acetate, a first half amount of said polyethylene glycol 400 and said propylene glycol with a predetermined amount of anhydrous ethanol to form said first mixture, and heating said first mixture to dissolve, wherein the step (b) further comprises the steps of:
(b.1) forming a second mixture by rapidly stirring said Carbopol 980NF and slowly adding with deionized water until said second mixture fully swells;
(b.2) adding another half amount of said polyethylene glycol 400 and said propylene glycol into said second mixture to form a third mixture, and stirring said third mixture uniformly;
(b.3) adding and stirring a predetermined amount of ethanol solution of triethanolamine with said third mixture to form a clear, transparent Carbopol gel;
(b.4) slowly adding said first mixture into said clear, transparent Carbopol gel, and adding and stirring said ethanol to prescribed weight to form said final product.

17. The method, as recited in claim 16, wherein 200 g of Bifonazole; 1 g of Triamcinolone acetonide acetate, 150 g of Carbopol 980NF, 2000 g of 1,2-propanediol, 2400 g of deionized water, 3000 g of Polyethylene glycol 400 (PEG 400), appropriate amount of Triethanolamine, and Ethanol are used to make 1000 units of said final product in gel form, wherein each unit of said final product contains 200 mg of Bifonazole and 1 mg of Triamcinolone acetonide acetate.

18. The method, as recited in claim 11, wherein said pharmaceutically acceptable accessory comprises Stearic acid, Glyceryl monostearate, Glycerol, White Vaseline, and Ethyl Hydroxybenzoate.

19. The method, as recited in claim 18, wherein the step (a) further comprises a step of mixing said Bifonazole and said Triamcinolone acetonide acetate with a mixture of said glycerol, said white Vaseline, and said ethyl hydroxybenzoate to form said first mixture, and heating said first mixture until its dissolves, wherein the step (b) further comprises the steps of:
(b.1) mixing said stearic acid and said glyceryl monostearate to form a second mixture, and heating up said second mixture at 80° C. and maintaining its temperature;
(b.2) adding and stirring said first mixture into said second mixture to form a cream form mixture; and
(b.3) adding water into said cream form mixture and continuously stirring said cream form mixture until said cream form mixture is cooled down to form said final product in cream form.

20. The method, as recited in claim 19, wherein 1 g of Bifonazole; 50 g of Triamcinolone acetonide acetate; 1000 g of stearic acid, 1000 g of Glyceryl monostearate, 1500 g of Glycerol, 800 g of white Vaseline, appropriate amount of Ethyl Hydroxybenzoate, and water are used to make 1000 units of said final product in gel form, wherein each unit of said final product contains 1 mg of Bifonazole and 50 mg of Triamcinolone acetonide acetate.
